(19) United States
(12) Patent Application Publication
(10) Pub. No.: US 2021/0282656 A1
(43) Pub. Date: Sep. 16, 2021

(54) BLOOD PRESSURE MONITORING METHOD, APPARATUS, AND DEVICE

(71) Applicant: HUAWEI TECHNOLOGIES CO., LTD., Shenzhen (CN)

(72) Inventors: Jing LI, Shenzhen (CN); Yu ZHU, Shenzhen (CN); Wenjuan CHEN, Shenzhen (CN)

(21) Appl. No.: 16/497,460

(22) PCT Filed: Apr. 24, 2017
(86) PCT No.: PCT/CN2017/081734

§ 371 Date: Sep. 25, 2019

(30) Foreign Application Priority Data
Apr. 1, 2017 (CN) 201710213584.4

(51) Int. Cl. A61B 5/021; A61B 5/00

(52) U.S. Cl. CPC A61B 5/02108; A61B 5/318; A61B 5/7267

(57) ABSTRACT

A blood pressure monitoring method, apparatus, and device are provided. The method includes: collecting a first biological signal of a to-be-measured user (S101); and predicting a first blood pressure value of the to-be-measured user based on the first biological signal and a pre-established individual calibration model (102). According to the method, only the first biological signal of the to-be-measured user needs to be collected to predict the first blood pressure value of the to-be-measured user. A collection manner is simple, and sleep of the user is not interrupted, greatly improving user experience.

### TECHNICAL FIELD

This application relates to the communications technology, and in particular, to a blood pressure monitoring method, apparatus, and device.

### BACKGROUND

Blood pressure is power of driving blood to cyclically flow in blood vessels, and can provide a sufficient blood volume for tissues and organs, to maintain normal metabolism of the organs. Hypertension with a symptom of increase of blood pressure is a very common cardiovascular disease, and hypertension causes a lot of harms such as cerebral apoplexy, blindness, and myocardial infarction. Blood pressure of a human body varies during one day, and factors such as emotion, exercise, eating, smoking, and drinking all affect the blood pressure. Therefore, occasional blood pressure measurement has relatively high contingency. Compared with the occasional blood pressure monitoring, continuous blood pressure monitoring (where to be specific, a blood pressure value is measured at a particular time interval within a time period) can improve an early diagnosis of hypertension, to better prevent cardiovascular complications and predict occurrence and progression of hypertensive complications and death.

Currently, a common manner of continuous blood pressure monitoring is continuously monitoring blood pressure through pressurization and air inflation on a cuff, and the manner is essentially use of a cuff blood pressure meter. The blood pressure is usually measured based on an oscillation method. A specific process is: A blood pressure value is measured through pressurization and air inflation on a cuff at a particular time interval, and then each measurement result is manually recorded.

However, in the blood pressure monitoring method in the prior art, air inflation and air deflation need to be frequently performed on the cuff, resulting in relatively poor user experience. In addition, when a user falls asleep, the air inflation on the cuff interrupts normal sleep of the user, and noise generated due to the air inflation on the cuff causes a heart rate and blood pressure of the user to increase. Therefore, the blood pressure monitoring method in the prior art cannot be applied to blood pressure monitoring at night.

### SUMMARY

This application provides a blood pressure monitoring method, apparatus, and device, to resolve technical problems in the prior art that user experience is relatively poor when continuous blood pressure monitoring is performed on a user through pressurization and air inflation on a cuff and a blood pressure monitoring method in the prior art cannot be applied to blood pressure monitoring at night because the air inflation on the cuff interrupts normal sleep of the user when the user falls asleep.

According to a first aspect, this application provides a blood pressure monitoring method, including:

collecting a first biological signal of a to-be-measured user; and

predicting a first blood pressure value of the to-be-measured user based on the first biological signal and a pre-established individual calibration model, where

the individual calibration model is obtained based on calibration data of the to-be-measured user and preset model training data; the calibration data includes a second blood pressure value actually measured before the first biological signal of the to-be-measured user is collected and a second biological signal corresponding to the second blood pressure value; the model training data includes actually-measured third blood pressure values of training users and third biological signals corresponding to the third blood pressure values; and the first biological signal, the second biological signal, and the third biological signals are all physiological signals that can generate waveforms.

According to the method provided in the first aspect, a blood pressure monitoring device only needs to collect the first biological signal of the to-be-measured user, to predict a first blood pressure value of the to-be-measured user at a current time point and/or in a time period in the future, thereby implementing continuous blood pressure monitoring. The first biological signal is a physiological signal that can generate a waveform. A collection manner of the first biological signal is simple; and there is no need to frequently perform air inflation and air deflation by using a cuff blood pressure meter, and therefore, there is no need to interrupt sleep of the user at night due to the frequent air inflation and air deflation, so that user experience is greatly improved, and the method provided in the first aspect can be applied to blood pressure monitoring at night. On the other hand, the individual calibration model in this application is obtained by using the calibration data of the to-be-measured user and the preset model training data. The calibration data reflects a real physical condition of the to-be-measured user, and physiological parameters of most users are gathered in the model training data, so that the individual calibration model can truly reflect an individual difference of the to-be-measured user. Therefore, precision of blood pressure prediction is greatly improved by using the individual calibration model in this application.

In a possible design, the method further includes:

obtaining at least one piece of calibration data of the to-be-measured user; and

establishing, based on the at least one piece of calibration data and the model training data, the individual calibration model corresponding to the to-be-measured user.

In a possible design, the method further includes:

obtaining at least one piece of new calibration data of the to-be-measured user when a preset model update period arrives; and

updating the individual calibration model of the to-be-measured user based on the at least one piece of new calibration data, to obtain a new individual calibration model.

In a possible design, the establishing, based on the at least one piece of calibration data and the model training data, the individual calibration model corresponding to the to-be-measured user includes:

determining, in the model training data based on the at least one piece of calibration data, a training data set required by the to-be-measured user; and

obtaining, based on the training data set required by the to-be-measured user and a preset modeling algorithm, the individual calibration model corresponding to the to-be-measured user, where the individual calibration model is a parameter set including a plurality of model parameters.

According to the methods provided in the foregoing possible designs, the at least one piece of calibration data of the to-be-measured user is obtained, and the individual calibration model corresponding to the to-be-measured user is established based on the at least one piece of calibration data and the model training data. The calibration data reflects the real physical condition of the to-be-measured user, and the physiological parameters of the most training users are gathered in the model training data, so that the individual calibration model can truly reflect the individual difference of the to-be-measured user. Therefore, the precision of the blood pressure prediction of the to-be-measured user is greatly improved by using the individual calibration model in this application. On the other hand, in this embodiment, the individual calibration model of the to-be-measured user can be periodically updated with reference to the new calibration data of the to-be-measured user, to predict the first blood pressure value of the to-be-measured user based on the new individual calibration model, thereby further improving the precision of the blood pressure prediction.

In a possible design, the predicting a first blood pressure value of the to-be-measured user based on the first biological signal and a preset individual calibration model specifically includes:

performing a feature extraction operation on the first biological signal, to obtain a feature set that can represent the first biological signal, where the feature set includes feature values arranged in a preset feature sequence, and feature values in different sequences represent different features of the first biological signal; and

calculating the feature values in the feature set and the model parameters in the parameter set based on a preset algorithm, to obtain the first blood pressure value of the to-be-measured user.

In a possible design, the collecting a first biological signal of a to-be-measured user specifically includes:

determining whether the to-be-measured user is in a motionless state; and

collecting the first biological signal of the to-be-measured user in a preset collection period when the to-be-measured user is in a motionless state and wears a blood pressure monitoring device.

In a possible design, the first biological signal, the second biological signal, and the third biological signals are all pulse wave signals of the to-be-measured user.

According to the method provided in the foregoing possible designs, the feature extraction is performed on the collected first biological signal, to obtain the feature set that can represent the first biological signal, and each feature value in the feature set is used as an input value of the individual calibration model. The individual calibration model is essentially a group of parameters. Therefore, the blood pressure monitoring device can calculate the feature values in the feature set and the model parameters in the parameter set based on the preset algorithm, to obtain the first blood pressure value of the to-be-measured user. The individual calibration model is obtained based on the calibration data of the to-be-measured user and the model training data of the training users, and the individual calibration model can truly reflect the individual difference of the to-be-measured user. Therefore, when blood pressure of the to-be-measured user needs to be predicted, the blood pressure of the user can be predicted based only on the collected first biological signal, prediction precision is high, and a prediction manner is simple. In addition, the blood pressure monitoring device in this application integrates functions of blood pressure collection, biological signal collection, biological signal processing, model establishment, and blood pressure tracing, so that an apparatus is simpler and can be more conveniently used by the user, reducing complexity of a wearable continuous blood pressure monitoring apparatus, and improving user experience on blood pressure monitoring. Further, the blood pressure monitoring device in this application can be automatically triggered to collect data of the blood pressure and the biological signal, in other words, the blood pressure monitoring device can easily obtain the model training data, to implement continuous blood pressure monitoring and household monitoring.

In a possible design, the method further includes:

generating a blood pressure variation curve based on first blood pressure values predicted at different time points; and

displaying the blood pressure variation curve.

According to the method provided in the possible design, the to-be-measured user can learn of a blood pressure variation status of the user within a time period, to adjust, with reference to exercise and diets of the user, life factors affecting the blood pressure, thereby providing an effective reference and basis for proper blood pressure control of the to-be-measured user.

In a possible design, the method further includes:

outputting prompt information when the first blood pressure value of the to-be-measured user is greater than a preset threshold, where the prompt information is used to inform that blood pressure is abnormal.

According to the method provided in the possible design, the to-be-measured user, or a family member or a friend of the to-be-measured user, can learn of an abnormal blood pressure status of the to-be-measured user in time, so that the to-be-measured user can avoid, in time, a problem of hypertensive complications caused by excessively high blood pressure.

In a possible design, the method further includes:

obtaining a period setting operation entered by the to-be-measured user;

displaying a period setting screen based on the period setting operation, where the period setting screen includes a plurality of model update periods; and

obtaining the preset model update period based on a period selection operation of the to-be-measured user on the period setting screen.

According to the method provided in the possible design, the blood pressure monitoring device may display the period setting screen to the user, so that the user can select a model update period suitable for the user based on the period setting screen, thereby improving intelligence of interaction between the user and the blood pressure monitoring device, satisfying a use requirement of the user, and improving the experience effect of the user.

According to a second aspect, to implement the blood pressure monitoring method in the first aspect, an embodiment of this application provides a blood pressure monitoring device. The device has a function of implementing the blood pressure monitoring method. The function may be implemented by hardware, or may be implemented by hardware executing corresponding software. The hardware or software includes one or more modules corresponding to the foregoing function.

In a possible implementation of the second aspect, the blood pressure monitoring device includes a plurality of function modules or units, configured to implement any blood pressure monitoring method according to the first aspect.

In another possible implementation of the second aspect, a structure of the blood pressure monitoring device may include a processor and a collector. The processor is configured to support the device in performing a corresponding function in any blood pressure monitoring method according to the first aspect. The collector is configured to collect a corresponding biological signal or blood pressure, so that the processor can predict blood pressure of the user based on collected data. The device may further include a memory. The memory is configured to couple to the processor, and stores a program instruction and data necessary for the blood pressure monitoring device to perform the blood pressure monitoring method.

According to a third aspect, an embodiment of this application provides a computer storage medium, configured to store a computer software instruction used by the foregoing blood pressure monitoring device. The computer software instruction includes a program designed for executing the first aspect.

According to a fourth aspect, an embodiment of this application provides a computer program product, including an instruction. When the computer program is executed by a computer, the instruction enables the computer to perform a function performed by a blood pressure monitoring device in the foregoing method.

Compared with the prior art, according to the blood pressure monitoring method, apparatus, and device provided in this application, the blood pressure monitoring device only needs to collect the first biological signal of the to-be-measured user, to predict the first blood pressure value of the to-be-measured user at the current time point and/or in the time period in the future, thereby implementing the continuous blood pressure monitoring. The first biological signal is a physiological signal that can generate a waveform. The collection manner of the first biological signal is simple, and there is no need to frequently perform the air inflation and air deflation by using the cuff blood pressure meter, and therefore, there is no need to interrupt the sleep of the user at night due to the frequent air inflation and air deflation, so that the experience effect of the user is greatly improved, and the blood pressure monitoring method can be applied to the blood pressure monitoring at night. On the other hand, the individual calibration model in this application is obtained by using the calibration data of the to-be-measured user and the preset model training data. The calibration data reflects the real physical condition of the to-be-measured user, and the physiological parameters of the most users are gathered in the model training data, so that the individual calibration model can truly reflect the individual difference of the to-be-measured user. Therefore, the precision of the blood pressure prediction is greatly improved by using the individual calibration model in this application.

### BRIEF DESCRIPTION OF DRAWINGS

FIG. 1 is a block diagram of a blood pressure monitoring device according to this application;

FIG. 2 is a schematic flowchart of Embodiment 1 of a blood pressure monitoring method according to this application;

FIG. 3 is a schematic flowchart of Embodiment 2 of a blood pressure monitoring method according to this application;

FIG. 4 is a schematic flowchart of Embodiment 3 of a blood pressure monitoring method according to this application;

FIG. 5 is a schematic flowchart of Embodiment 4 of a blood pressure monitoring method according to this application;

FIG. 6 is a schematic flowchart of Embodiment 5 of a blood pressure monitoring method according to this application;

FIG. 7 is a schematic flowchart of Embodiment 6 of a blood pressure monitoring method according to this application;

FIG. 8 is a schematic structural diagram of Embodiment 1 of a blood pressure monitoring apparatus according to this application;

FIG. 9 is a schematic structural diagram of Embodiment 2 of a blood pressure monitoring apparatus according to this application;

FIG. 10 is a schematic structural diagram of Embodiment 3 of a blood pressure monitoring apparatus according to this application;

FIG. 11 is a schematic structural diagram of Embodiment 4 of a blood pressure monitoring apparatus according to this application; and

FIG. 12 is a schematic structural diagram of an embodiment of a blood pressure monitoring device according to the present invention.

### DESCRIPTION OF EMBODIMENTS

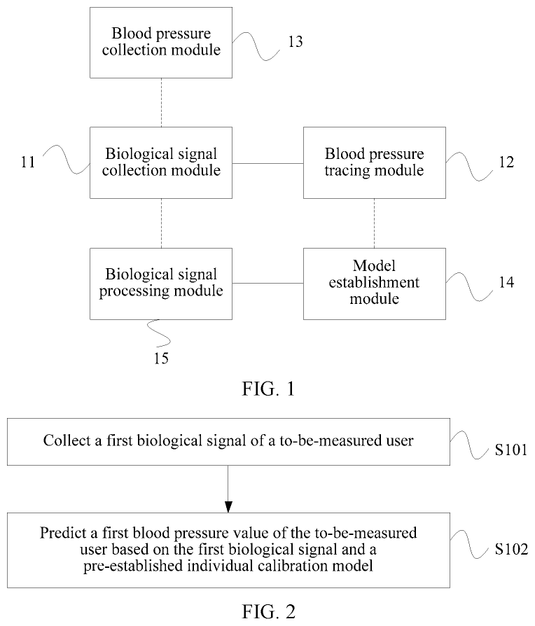

A blood pressure monitoring method, apparatus, and a device provided in embodiments of this application may be applied to a scenario of human-body blood pressure monitoring. Optionally, an execution body of the blood pressure monitoring method may be a blood pressure monitoring device. The blood pressure monitoring device may be a terminal device having a blood pressure monitoring function, or may be a wearable device having a blood pressure monitoring function. The wearable device may be a device worn around the arm or the wrist, or may be a device worn in the chest or in the palm, or may be a device worn on the head. A specific form of the wearable device is not limited in this application. Optionally, the blood pressure monitoring device may be divided into a plurality of modules based on functions. As shown in FIG. 1, the blood pressure monitoring device may include a biological signal collection module 11 and a blood pressure tracing module 12. Optionally, the blood pressure monitoring device may further include a blood pressure collection module 13, a model establishment module 14, and a biological signal processing module 15. For a function of each module or an operation performed by each module and a connection relationship between the modules, refer to descriptions in the following embodiments.

In the prior art, during continuous blood pressure monitoring of a user, the continuous blood pressure monitoring is usually performed through pressurization and air inflation on a cuff. However, in this blood pressure monitoring method in the prior art, air inflation and air deflation need to be frequently performed on the cuff, resulting in relatively poor user experience. Particularly, when the user falls asleep, the air inflation on the cuff interrupts normal sleep of the user, and noise generated due to the air inflation on the cuff causes a heart rate and blood pressure of the user to increase. Therefore, the blood pressure monitoring method cannot be applied to blood pressure monitoring at night. The blood pressure monitoring method and device provided in this application aim to resolve the foregoing technical problems in the prior art.

It should be noted that terms used in the embodiments of this application are merely for the purpose of illustrating specific embodiments, and are not intended to limit this application. The terms “a”, “said” and “the” of singular forms used in the embodiments and the appended claims of this application are also intended to include plural forms, unless otherwise specified in the context clearly. It should also be understood that, the term “and/or” used herein indicates and includes any or all possible combinations of one or more associated listed items. The character “/” in this specification generally indicates an “or” relationship between associated objects.

It should be understood that although terms such as “first”, “second”, and “third” may be used in the embodiments of this application to describe various messages, requests, and terminals, these messages, requests, and terminals should not be limited to these terms. These terms are merely used to distinguish the messages, requests, and terminals. For example, a first terminal may also be referred to as a second terminal without departing from the scope of the embodiments of this application. Similarly, a second terminal may also be referred to as a first terminal.

Depending on the context, for example, the word “if” used herein may be explained as “while” or “when” or “in response to determining” or “in response to detection”. Similarly, depending on the context, the phrase “if determining” or “if detecting (a stated condition or event)” may be explained as “when determining” or “in response to determining” or “when detecting (the stated condition or event)” or “in response to detecting (the stated condition or event)”.

Technical solutions in this application and how to resolve the foregoing technical problems by using the technical solutions in this application are described in detail below by using specific embodiments. The following several specific embodiments may be combined with each other, and a same or similar concept or process may not be described repeatedly in some embodiments. The following describes the embodiments of this application with reference to accompanying drawings.

FIG. 2 is a schematic flowchart of Embodiment 1 of a blood pressure monitoring method according to this application. This embodiment relates to a specific process in which a blood pressure monitoring device collects a biological signal of a to-be-measured user and predicts blood pressure of the to-be-measured user at one or more time points in the future based on the collected biological signal and a preset individual calibration model, to implement blood pressure monitoring of the to-be-measured user. As shown in FIG. 2, the method includes the following steps.

S101. Collect a first biological signal of the to-be-measured user. Specifically, when the to-be-measured user wears the blood pressure monitoring device and starts the blood pressure monitoring device, the blood pressure monitoring device can collect the first biological signal of the to-be-measured user. Optionally, the first biological signal is a physiological signal of a human body that can generate a waveform. For example, the first biological signal may be an electrocardiogram signal or an electroencephalogram signal, or may even be a respiratory rate of the human body. A specific form of the first biological signal is not limited in this application, provided that the first biological signal is a physiological signal that is generated by the human body and that has a specific waveform. Optionally, step S101 may be obtained by using the biological signal collection module shown in FIG. 1.

S102. Predict a first blood pressure value of the to-be-measured user based on the first biological signal and a pre-established individual calibration model.

The individual calibration model is obtained based on calibration data of the to-be-measured user and preset model training data; the calibration data includes a second blood pressure value actually measured before the first biological signal of the to-be-measured user is collected and a second biological signal corresponding to the second blood pressure value; the model training data includes actually-measured third blood pressure values of training users and third biological signals corresponding to the third blood pressure values; and the first biological signal, the second biological signal, and the third biological signals are all physiological signals that can generate waveforms.

Specifically, in this application, an individual calibration model is preset in the blood pressure monitoring device. The individual calibration model may be obtained by using the calibration data of the to-be-measured user and the preset model training data after the to-be-measured user obtains a delivered blood pressure monitoring device, or may be an individual calibration model obtained by the to-be-measured user by performing model updating based on a physical condition of the to-be-measured user after the to-be-measured user uses the blood pressure monitoring device for a period of time. Optionally, the individual calibration model may be obtained by the blood pressure monitoring device by using a corresponding modeling method, or may be obtained by the blood pressure monitoring device from another model establishment device (for example, a computer). Optionally, the individual calibration model may be obtained by training the model training data and the calibration data of the to-be-measured user by using regression methods such as linear regression and support vector machine. The modeling method is not limited in this application.

On one hand, the calibration data of the to-be-measured user includes the second blood pressure value actually measured before the first biological signal of the to-be-measured user is collected and the second biological signal corresponding to the second blood pressure value. There may be one or more pieces of calibration data, and this application is not limited thereto. The second biological signal is also a physiological signal of a human body that can generate a waveform, and a type of the second biological signal may be the same as that of the first biological signal. The calibration data may be directly obtained by the blood pressure monitoring device through measurement, or may be obtained by the blood pressure monitoring device by using another device that can wiredly or wirelessly communicate with the blood pressure monitoring device. It should be noted that, that “the second blood pressure value corresponds to the second biological signal” herein actually means that a measurement time point of the second blood pressure value is the same as a collection time point of the second biological signal, or a time difference between the measurement time point and the collection time point is less than a preset threshold, so that the second blood pressure value is related to the second biological signal.

On the other hand, the model training data includes the actually-measured third blood pressure values of the training users and the third biological signals corresponding to the third blood pressure values. The model training data may be obtained by the blood pressure monitoring device by collecting, before delivery, third blood pressure values of a plurality of training users and third biological signals corresponding to the third blood pressure values. In other words, the model training data includes a plurality of third blood pressure values and a plurality of third biological signals. The third biological signals are also physiological signals of a human body that can generate a waveform, and types of the third biological signal may be the same as those of the first biological signal and the second biological signal. The model training data may be directly obtained by the blood pressure monitoring device through measurement, or may be obtained by the blood pressure monitoring device by using another device that can wiredly or wirelessly communicate with the blood pressure monitoring device. It should be noted that, that “the third blood pressure value corresponds to the third biological signal” herein actually means that a measurement time point of the third blood pressure value is the same as a collection time point of the third biological signal, or a time difference between the measurement time point and the collection time point is less than a preset threshold, so that the third blood pressure value is related to the third biological signal. Optionally, the training users may be users other than the to-be-measured user, or may be some users including the to-be-measured user. An individual type of the training users is not limited in this embodiment.

Therefore, after the blood pressure monitoring device collects the first biological signal of the to-be-measured user (where the first biological signal is collected by the foregoing biological signal collection module), the blood pressure monitoring device may correspondingly process the first biological signal into biological data matching an input format of the individual calibration model, so that the biological data is used as an input of the individual calibration model to predict the first blood pressure value of the to-be-measured user. Optionally, a first blood pressure value of the to-be-measured user at a specific time point may be preset (where for example, a first blood pressure value of the to-be-measured user at a current time point is predicted, or a first blood pressure value of the to-be-measured user at a specific time point in the future may be predicted); or a first blood pressure value of the to-be-measured user in a specific time period in the future may be predicted.

It should be noted that, the blood pressure monitoring device may periodically collect the first biological signal of the to-be-measured user. Therefore, each time the first biological signal is collected, a first blood pressure value of the to-be-measured user at a specific time point or in a specific time period may be predicted based on the preset individual calibration model. There is a specific correspondence between the collection time point of the first biological signal and a blood pressure prediction time. For example, if the blood pressure monitoring device collects a first biological signal of the to-be-measured user at 9:00 in the morning, the blood pressure monitoring device predicts a first blood pressure value of the to-be-measured user from 9:00 to 10:00 in the morning based on the first biological signal collected at 9:00; then, if the blood pressure monitoring device collects a first biological signal of the to-be-measured user at 9:30 again, the blood pressure monitoring device predicts a first blood pressure value of the to-be-measured user from 9:30 to 10:30 in the morning based on the first biological signal collected at 9:30. Based on the foregoing descriptions, the blood pressure monitoring device may obtain a plurality of first blood pressure values of the to-be-measured user at different time points, to complete continuous blood pressure monitoring of the to-be-measured user. Optionally, step 5102 may be performed by the blood pressure tracing module shown in FIG. 1.

It may be learned from the foregoing descriptions that, according to the blood pressure monitoring method provided in this application, the blood pressure monitoring device only needs to collect the first biological signal of the to-be-measured user, to predict the first blood pressure value of the to-be-measured user at the current time point and/or in the time period in the future, thereby implementing the continuous blood pressure monitoring. The first biological signal is a physiological signal that can generate a waveform. A collection manner of the first biological signal is simple; and there is no need to frequently perform air inflation and air deflation by using a cuff blood pressure meter, and therefore, there is no need to interrupt sleep of the user at night due to the frequent air inflation and air deflation, so that user experience is greatly improved, and the blood pressure monitoring method can be applied to blood pressure monitoring at night. On the other hand, the individual calibration model in this application is obtained by using the calibration data of the to-be-measured user and the preset model training data. The calibration data reflects a real physical condition of the to-be-measured user, and physiological parameters of most users are gathered in the model training data, so that the individual calibration model can truly reflect an individual difference of the to-be-measured user. Therefore, precision of blood pressure prediction is greatly improved by using the individual calibration model in this application.

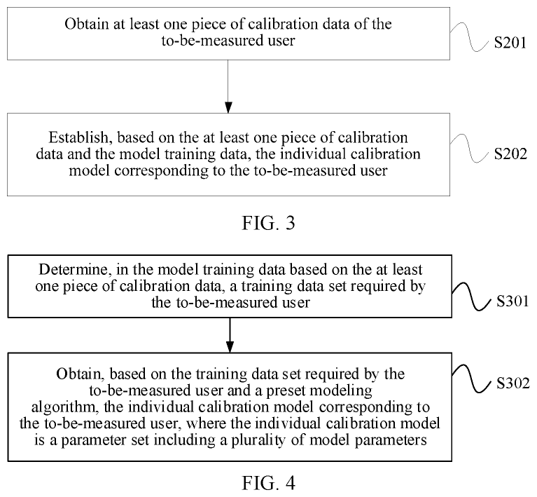

FIG. 3 is a schematic flowchart of Embodiment 2 of a blood pressure monitoring method according to this application. This embodiment relates to a specific process in which a blood pressure monitoring device autonomously collects calibration data of a to-be-measured user and establishes, by using the collected calibration data and the foregoing preset model training data, an individual calibration model corresponding to the to-be-measured user. It should be noted herein that, in this application, each to-be-measured user has different calibration data (where each blood pressure monitoring device may have same or different model training data). Therefore, each to-be-measured user corresponds to a different individual calibration model. Continuing to refer to a structural diagram of the blood pressure monitoring device shown in FIG. 1, in this embodiment, the blood pressure monitoring device may further include a blood pressure collection module, a model establishment module, and a biological signal processing module in addition to the foregoing biological signal collection module and blood pressure tracing module. Based on the foregoing embodiment, further, before step 5101, the method may further include the following steps.

S201. Obtain at least one piece of calibration data of the to-be-measured user.

Specifically, the step may be performed by the blood pressure collection module of the blood pressure monitoring device. The blood pressure collection module is mainly configured to obtain a calibration blood pressure value (where the calibration blood pressure value is the foregoing second blood pressure value, namely, a blood pressure value actually measured by the blood pressure collection module) of the to-be-measured user. The blood pressure collection module is connected to the biological signal collection module. It should be noted that, in this embodiment, the blood pressure monitoring device is a wearable device worn around the arm or the wrist of the to-be-measured user. When the user uses a blood pressure collection function in the wearable mobile device, the wearable device pushes some suggested configurations to the to-be-measured user, so that the user selects a blood pressure measurement time point (where the user may select a plurality of blood pressure measurement time points). After the user selects and saves the measurement time point, each time a specified measurement point (namely, the blood pressure measurement time point) arrives, the blood pressure collection module obtains, based on a confirmation of the to-be-measured user, the calibration blood pressure value (namely, the second blood pressure value) of the to-be-measured user, and records the current second blood pressure value and the current blood pressure measurement time point.

In a specific implementation process, the wearable device may be a micropump blood pressure watch. The blood pressure collection module may include a built-in micropump, a wrist strap used for blood pressure measurement, and a pressure sensor. A collection process of the calibration blood pressure value is specifically: The micropump blood pressure watch automatically inflates air into the wrist strap through pressurization by using the micropump, stops the pressurization after the air is inflated for a specific time, and starts to deflate the air. When atmospheric pressure is decreased to a specific extent, a blood flow can pass through blood vessels and has a specific oscillatory wave. The oscillatory wave is propagated to the pressure sensor. The pressure sensor can detect, in real time, pressure and fluctuation in the customized wrist strap, and then measures and calculates the calibration blood pressure value (namely, the second blood pressure value) by using a particular algorithm based on the pressure and the fluctuation.

The blood pressure collection module is connected to the biological signal collection module, and as described in Embodiment 1, the biological signal collection module is configured to collect the second biological signal of the to-be-measured user, and provide the second biological signal to the biological signal processing module connected to the biological signal collection module, to perform corresponding processing. Each time after the blood pressure value is obtained through the pressurization on the wrist strap and air deflation is completed, the wearable device automatically collects the first biological signal of the user for one to two minutes by using the biological signal collection module. The second biological signal collected by the biological signal collection module and the second blood pressure value actually measured by the blood pressure collection module are combined together as one piece of calibration data of the to-be-measured user. According to the method, a plurality of pieces of calibration data of the to-be-measured user can be obtained.

Optionally, in this embodiment, the first biological signal, the second biological signal, and the third biological signals are pulse wave signals of the to-be-measured user or the training users, and the blood pressure monitoring device (namely, the wearable device in this embodiment) may be used as a carrier of the biological signal collection module and the blood pressure collection module.

S202. Establish, based on the at least one piece of calibration data and the model training data, the individual calibration model corresponding to the to-be-measured user. Specifically, the step may be cooperatively performed by the biological signal processing module and the model establishment module, and the biological signal processing module is separately connected to the biological signal collection module and the model establishment module.

After obtaining the at least one piece of calibration data that is jointly collected by the biological signal collection module and the blood pressure collection module, the biological signal processing module processes one or more second biological signals in all the calibration data that is jointly collected by the biological signal collection module and the blood pressure collection module, to obtain a feature set that can represent the second biological signals. It should be noted that, each second biological signal corresponds to a group of feature values that can represent the second biological signal. For example, assuming that the second biological signal is a pulse wave signal, feature values that can represent the pulse wave signal may be values such as a peak value, a valley value, and a time difference between a peak and a valley of the pulse wave signal (namely, a period of the pulse wave signal).

In addition, in this embodiment, the third blood pressure values in the model training data and the third biological signals corresponding to the third blood pressure values may also be actually measured or collected by the blood pressure collection module and the biological signal collection module of the wearable device in this embodiment.

Further, the individual calibration model corresponding to the to-be-measured user may be obtained by using a corresponding modeling algorithm based on each second blood pressure value in all the calibration data jointly collected by the biological signal collection module and the blood pressure collection module and the feature set of the second biological signal corresponding to each second blood pressure value. Optionally, the obtained individual calibration model may be a parameter set including a plurality of model parameters.

Optionally, in a possible implementation of step 5202, referring to Embodiment 3 shown in FIG. 4, a specific process of the establishing the individual calibration model of the to-be-measured user may include the following steps.

S301. Determine, in the model training data based on the at least one piece of calibration data, a training data set required by the to-be-measured user.

S302. Obtain, based on the training data set required by the to-be-measured user and a preset modeling algorithm, the individual calibration model corresponding to the to-be-measured user, where the individual calibration model is a parameter set including a plurality of model parameters.

With reference to step 5301 and step 5302, the two steps are performed by the model establishment module in FIG. 1. Before the individual calibration model of the to-be-measured user is established, the blood pressure collection module and the biological signal collection module obtain sufficient third blood pressure values and third biological signals corresponding to the third blood pressure values. The biological signal processing module performs feature extraction to obtain feature sets that can represent the third biological signals. The feature set of each third biological signal includes a plurality of feature values that can represent features of the third biological signal. The feature set of each third biological signal and the third blood pressure value corresponding to the third biological signal form the model training data.

After the wearable device obtains sufficient model training data, the wearable device may be delivered from a factory. After delivery, assuming that the wearable device is purchased by the to-be-measured user, after the to-be-measured user enables a blood pressure monitoring function of the wearable device, the model establishment module of the wearable device starts to work, to be specific, the model establishment module triggers the blood pressure collection module and the biological signal processing module to collect the at least one piece of calibration data of the to-be-measured user and determines, in the model training data based on one or more second blood pressure values in all the calibration data, the training data set required by the to-be-measured user. For example, if an average value of second blood pressure values of calibration data used by a user is 130, a part in which blood pressure values (namely, third blood pressure values) are approximate to 130 (for example, within an interval from 120 to 140) in the model training data is selected and used as a training data set of the user, and then an individual calibration model of the to-be-measured user is established based on the obtained calibration data and the training data set. Optionally, the model establishment module may perform model establishment by using regression methods such as linear regression and support vector machine. In this embodiment, the individual calibration model is essentially a group of parameters, in other words, an individual calibration module is a parameter set including a plurality of model parameters.

Optionally, in a model establishment process, a feature suitable for model establishment needs to be determined. The feature suitable for the model establishment may be sifted out by using an automatic feature selection method. The automatic feature selection method includes filter feature selection methods such as Pearson's correlation coefficient and information gain, wrapper feature selection methods such as sequential forward search and sequential floating forward search, or a feature selection method integrating the filter feature selection method and the wrapper feature selection method.

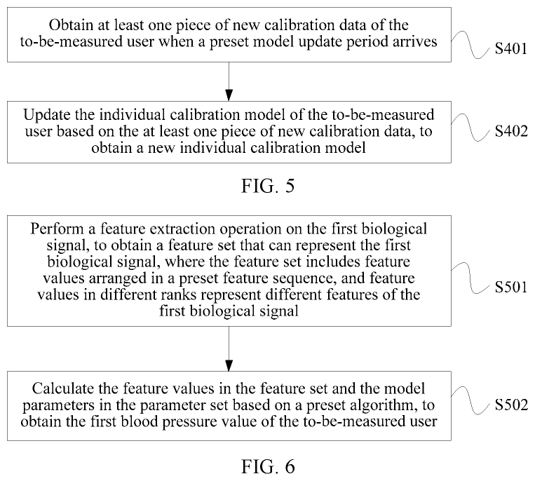

Optionally, the model establishment module may maintain the individual calibration model after establishing the individual calibration model. That is, during subsequent blood pressure prediction, the wearable device keeps using the individual calibration model. Optionally, the model establishment module may alternatively continuously update the individual calibration model based on an actual use process. For example, when a physical condition of the to-be-measured user changes or the to-be-measured user is directly changed, the individual calibration model needs to be updated, to ensure precision of subsequent blood pressure prediction. Referring to Embodiment 4 shown in FIG. 5, a process of updating the individual calibration model includes the following steps.

S401. Obtain at least one piece of new calibration data of the to-be-measured user when a preset model update period arrives.

S402. Update the individual calibration model of the to-be-measured user based on the at least one piece of new calibration data, to obtain a new individual calibration model.

With reference to step 5401 and step 5402, a model update period is preset in the model establishment module. For example, the individual calibration module of the to-be-measured user is updated every few days, or the individual calibration model of the to-be-measured user is updated every few hours. Therefore, when a model update period arrives, the model establishment module triggers the blood pressure collection module and the biological signal collection module to further obtain the at least one piece of new calibration data, and then updates, based on the at least one piece of new calibration data, the old individual calibration model of the to-be-measured user that is established before, to obtain the new individual calibration model. Optionally, the model establishment module may directly construct the new individual calibration model by using the method in step S201 to step S302 based on the at least one piece of new calibration data and the model training data. Alternatively, the model establishment module may obtain the new individual calibration model based on the at least one piece of new calibration data, the model training data, and all the calibration data that is collected before the previous individual calibration model of the to-be-measured user is established. For a specific model establishment process, refer to the method in step S201 to step S302, and details are not described herein again.

According to the method provided in this embodiment of this application, the at least one piece of calibration data of the to-be-measured user is obtained, and the individual calibration model corresponding to the to-be-measured user is established based on the at least one piece of calibration data and the model training data. The calibration data reflects the real physical condition of the to-be-measured user, and the physiological parameters of the most training users are gathered in the model training data, so that the individual calibration model can truly reflect the individual difference of the to-be-measured user. Therefore, the precision of the blood pressure prediction of the to-be-measured user is greatly improved by using the individual calibration model in this application. On the other hand, in this embodiment, the individual calibration model of the to-be-measured user can be periodically updated with reference to the new calibration data of the to-be-measured user, to predict the first blood pressure value of the to-be-measured user based on the new individual calibration model, thereby further improving the precision of the blood pressure prediction.

FIG. 6 is a schematic flowchart of Embodiment 5 of a blood pressure monitoring method according to this application. This embodiment relates to a specific process in which a wearable device predicts a first blood pressure value of a to-be-measured user based on a first biological signal collected by a blood pressure collection module and an individual calibration model established by a model establishment module. Based on the foregoing embodiments, step 5101 may specifically include the following steps.

S501. Perform a feature extraction operation on the first biological signal, to obtain a feature set that can represent the first biological signal, where the feature set includes feature values arranged in a preset feature sequence, and feature values in different sequences represent different features of the first biological signal.

Specifically, the step may be performed by the biological signal processing module. After collecting the first biological signal of the to-be-measured user, the biological signal collection module transmits the first biological signal to the biological signal processing module, so that the biological signal processing module performs the feature extraction operation on the first biological signal, that is, extracts relevant feature data that can represent the first biological signal (where optionally, the feature data may be denoted by x0, x1, x2, . . . , and xn). The relevant feature data is the feature set of the first biological signal. The foregoing feature extraction process is actually converting the biological signal into a group of specific feature values. These specific feature values are arranged in the preset feature sequence in the feature set, and the feature values in different sequences represent the different features of the first biological signal. For example, assuming that the feature set obtained by the biological signal processing module by performing the feature extraction operation on the first biological signal is {1, −1, 0.5}, and the preset feature sequence for arrangement in a system is {a peak, a valley, a time difference between the peak and the valley}, a feature value 1 in the feature set is a value of the peak, −1 is a value of the valley, and 0.5 is the time the distance between the peak and the valley. In a subsequent blood pressure prediction process, the blood pressure tracing module performs calculation by using the feature values of the first biological signal.

Optionally, the biological signal processing module may perform a filtering operation such as filtering on the first biological signal, that is, filter out noise or interference of the first biological signal, and then extract, from the filtered first biological signal, the relevant feature data that can represent the first biological signal, to ensure accuracy of feature extraction.

S502. Calculate the feature values in the feature set and the model parameters in the parameter set based on a preset algorithm, to obtain the first blood pressure value of the to-be-measured user.

Specifically, the step may be performed by the blood pressure tracing module. The module is connected to the model establishment module, and predicts the blood pressure value of the user by using the first biological signal. For each user, after the biological signal collection module in the wearable device collects a first biological signal of the user, the biological signal processing module obtains relevant feature data (namely, a feature set) of the first biological signal, and then inputs the relevant feature data of the user into the model establishment module. At last, a blood pressure value of the user is predicted by using an individual calibration model established by the model establishment module.

As described in the foregoing embodiment, the individual calibration model of the to-be-measured user is actually a group of parameters (namely, the parameter set including the plurality of model parameters). When performing blood pressure prediction, the blood pressure tracing module performs an operation on the relevant feature data (namely, the feature values in the feature set) of the collected first biological signal and the group of parameters (namely, the individual calibration model) according to a specified rule (namely, the preset algorithm), to obtain the predicted blood pressure value.

In a specific implementation process, assuming that the individual calibration model is obtained through model establishment by using a linear regression method, in other words, the individual calibration model is actually a linear regression predictive model, the linear regression predictive model is essentially a group of parameters that is set as B, B is specifically {b0, b1, b2, . . . , bn}, and the feature set (for example, x0, x1, x2, . . . , xn) of the first biological signal is used as input values of the individual calibration model, a specific implementation of the blood pressure monitoring is performing multiplication and addition operations on the parameters B and corresponding values of value features corresponding to the input values, to obtain the predicted first blood pressure value, in other words, the first blood pressure value BP=b0*x0+b1*x1+b2*x2+ . . . +bn*xn.

Optionally, the blood pressure tracing module may further generate a blood pressure variation curve based on first blood pressure values predicted at different time points, and then display the blood pressure variation curve, so that the to-be-measured user can learn of a blood pressure variation status of the user within a time period, and adjust, with reference to exercise and diets of the user, life factors affecting the blood pressure, thereby providing an effective reference and basis for proper blood pressure control of the to-be-measured user.

Optionally, when the first blood pressure value of the to-be-measured user is greater than a preset threshold, the blood pressure tracing module may further output prompt information, and the prompt information is used to inform that the blood pressure is abnormal. Optionally, the prompt information may be information directly provided for the to-be-measured user, or may be information provided for a family member or a friend of the to-be-measured user. In other words, when the blood pressure tracing module determines that the first blood pressure value of the to-be-measured user is greater than the preset threshold, the communications module of the blood pressure monitoring device may send the prompt information to an electronic device of the family member or the friend of the to-be-measured user, so that these people can also learn of a blood pressure monitoring status of the to-be-measured user and help the to-be-measured user in time.

Optionally, when the biological signal collection module collects the first biological signal of the user, the biological signal collection module may determine whether the to-be-measured user is in a motionless state. When determining that the to-be-measured user is in a motionless state and wears the blood pressure monitoring device, the biological signal collection module may collect the first biological signal of the to-be-measured user in a preset collection period, to implement blood pressure tracing. As described in the foregoing optional manner, all the predicted first blood pressure values may be drawn into the blood pressure curve. When the blood pressure value becomes abnormal, the to-be-measured user and/or the family member of the to-be-measured user may be prompted in proper time.

According to the blood pressure monitoring method provided in this embodiment of this application, the feature extraction is performed on the collected first biological signal, to obtain the feature set that can represent the first biological signal, and each feature value in the feature set is used as the input value of the individual calibration model. The individual calibration model is essentially a group of parameters. Therefore, the blood pressure monitoring device can calculate the feature values in the feature set and the model parameters in the parameter set based on the preset algorithm, to obtain the first blood pressure value of the to-be-measured user. The individual calibration model is obtained based on the calibration data of the to-be-measured user and the model training data of the training users, and the individual calibration model can truly reflect the individual difference of the to-be-measured user. Therefore, when the blood pressure of the to-be-measured user needs to be predicted, the blood pressure of the user can be predicted based only on the collected first biological signal, prediction precision is high, and a prediction manner is simple. In addition, the blood pressure monitoring device in this application integrates functions of blood pressure collection, biological signal collection, biological signal processing, model establishment, and blood pressure tracing, so that an apparatus is simpler and can be more conveniently used by the user, reducing complexity of a wearable continuous blood pressure monitoring apparatus, and improving user experience on blood pressure monitoring. Further, the blood pressure monitoring device in this application can be automatically triggered to collect data of the blood pressure and the biological signal, in other words, the blood pressure monitoring device can easily obtain the model training data, to implement continuous blood pressure monitoring and household monitoring.

In the foregoing embodiment, the model establishment module may update the individual calibration model of the to-be-measured user when the preset model update period arrives. The preset model update period may be an update period having been built in at delivery of the blood pressure monitoring device or may be set by the user. Alternatively, the blood pressure monitoring device may have a plurality of model update periods, and the preset model update period is an update period selected by the user from the plurality of model update periods.

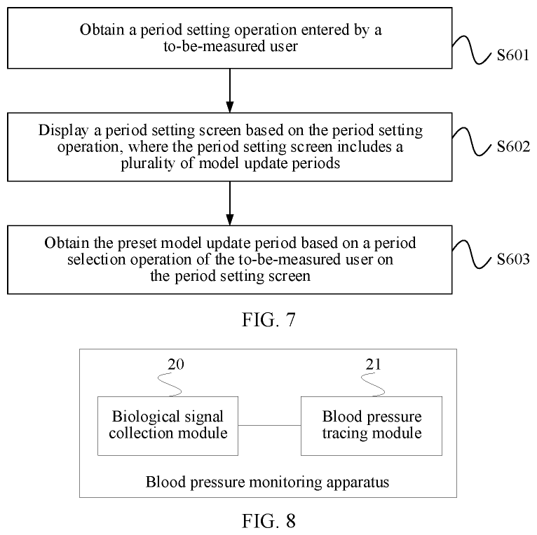

FIG. 7 is a schematic flowchart of Embodiment 6 of a blood pressure monitoring method according to this application. This embodiment relates to a specific process in which a blood pressure monitoring device obtains an actually used model update period based on a user setting. The method includes the following steps.

S601. Obtain a period setting operation entered by a to-be-measured user.

Specifically, the user may enter the period setting operation into the blood pressure monitoring device by touching or pressing a corresponding control based on the blood pressure monitoring device. Optionally, the blood pressure monitoring device may provide a trigger control for entering a period setting screen. The trigger control may be a virtual button or a physical button. The blood pressure monitoring device determines, based on a type and an operation of triggering the control by the user, that the period setting operation is entered by the to-be-measured user. Optionally, if the to-be-measured user taps a virtual control on a display interface of the blood pressure monitoring device, the blood pressure monitoring device may determine, based on coordinates at which the user taps the interface, whether the period setting operation is entered by the user.

S602. Display a period setting screen based on the period setting operation, where the period setting screen includes a plurality of model update periods.

Specifically, when the blood pressure monitoring device determines that an operation currently entered by the user is the period setting operation, the blood pressure monitoring device may display the period setting screen to the to-be-measured user, and the period setting screen includes the plurality of model update periods, for example, one day, two days, three days, and a week.

S603. Obtain the preset model update period based on a period selection operation of the to-be-measured user on the period setting screen.

Specifically, the to-be-measured user may perform selection based on a period display interface, that is, enter the period selection operation into the blood pressure monitoring device. The period selection operation may be an operation, such as tap, slide, or touch and hold, of the to-be-measured user on the period display interface. The blood pressure monitoring device may still determine, based on coordinates of the period selection operation of the user or other information, the model update period selected by the to-be-measured user. The model update period is the preset model update period used in the foregoing embodiment.

According to the method provided in this embodiment, the blood pressure monitoring device may display the period setting screen to the user, so that the user can select a model update period suitable for the user based on the period setting screen, thereby improving intelligence of interaction between the user and the blood pressure monitoring device, satisfying a use requirement of the user, and improving user experience.

FIG. 8 is a schematic structural diagram of Embodiment 1 of a blood pressure monitoring apparatus according to this application. The blood pressure monitoring apparatus may be implemented as a part of the foregoing blood pressure monitoring device or the entire blood pressure monitoring device by using software, hardware, or a combination of software and hardware. As shown in FIG. 8, the blood pressure monitoring apparatus may include a biological signal collection module 20 and a blood pressure tracing module 21.

Specifically, the biological signal collection module 20 is configured to collect a first biological signal of a to-be-measured user.

The blood pressure tracing module 21 is configured to predict a first blood pressure value of the to-be-measured user based on the first biological signal and a pre-established individual calibration model.

The individual calibration model is obtained based on calibration data of the to-be-measured user and preset model training data; the calibration data includes a second blood pressure value actually measured before the first biological signal of the to-be-measured user is collected and a second biological signal corresponding to the second blood pressure value; the model training data includes actually-measured third blood pressure values of training users and third biological signals corresponding to the third blood pressure values; and the first biological signal, the second biological signal, and the third biological signals are all physiological signals that can generate waveforms.

The blood pressure monitoring apparatus provided in this application can execute the foregoing method embodiment. Implementation principles and technical effects thereof are similar, and details are not described herein again.

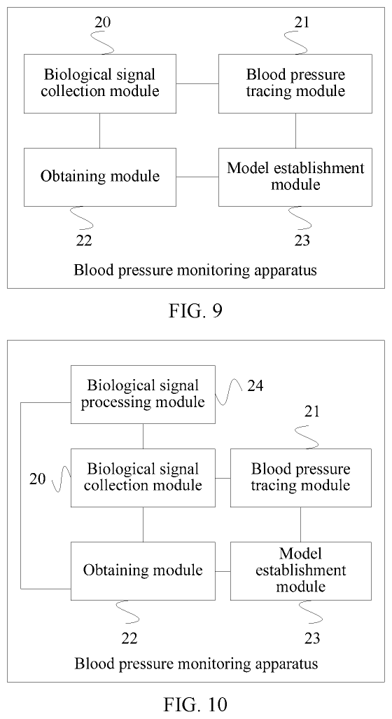

FIG. 9 is a schematic structural diagram of Embodiment 2 of a blood pressure monitoring apparatus according to this application. Based on the embodiment shown in FIG. 8, the apparatus further includes an obtaining module 22 and a model establishment module 23.

Optionally, the obtaining module 22 may include the foregoing biological signal collection module 20 and the blood pressure collection module 13 in the foregoing method embodiment. The blood pressure collection module 13 is configured to obtain the second blood pressure value actually measured before the first biological signal of the to-be-measured user is collected. The biological signal collection module 20 is further configured to obtain the second biological signal that is actually measured before the first biological signal of the to-be-measured user is collected and that corresponds to the second blood pressure value, to obtain at least one piece of calibration data based on the second blood pressure value and the second biological signal.

Optionally, the obtaining module 22 may alternatively be a module independent of the biological signal collection module 20 that is connected to the biological signal collection module 20, has a blood pressure collection function, and has a function of combining the second biological signal and the second blood pressure value into the at least one piece of calibration data. A specific division form of the obtaining module 22 is not limited in this embodiment. In a structure shown in FIG. 8, the obtaining module 22 is a module independent of the biological signal collection module 20 that is connected to the biological signal collection module 20.

The model establishment module 23 is configured to establish, based on the at least one piece of calibration data and the model training data, the individual calibration model corresponding to the to-be-measured user.

Further, the obtaining module 22 is further configured to obtain at least one piece of new calibration data of the to-be-measured user when a preset model update period arrives.

The model establishment module 23 is further configured to update the individual calibration model of the to-be-measured user based on the at least one piece of new calibration data, to obtain a new individual calibration model.

Still further, the model establishment module 23 is specifically configured to determine, in the model training data based on the at least one piece of calibration data, a training data set required by the to-be-measured user, and obtain, based on the training data set required by the to-be-measured user and a preset modeling algorithm, the individual calibration model corresponding to the to-be-measured user, where the individual calibration model is a parameter set including a plurality of model parameters.

The blood pressure monitoring apparatus provided in this application can execute the foregoing method embodiment. Implementation principles and technical effects thereof are similar, and details are not described herein again.

FIG. 10 is a schematic structural diagram of Embodiment 3 of a blood pressure monitoring apparatus according to this application. Based on the embodiment shown in FIG. 9, the apparatus further includes a biological signal processing module 24.

The biological signal processing module 24 is configured to perform a feature extraction operation on the first biological signal, to obtain a feature set that can represent the first biological signal. The feature set includes feature values arranged in a preset feature sequence, and feature values in different sequences represent different features of the first biological signal.

The blood pressure tracing module 21 is configured to calculate the feature values in the feature set and the model parameters in the parameter set based on a preset algorithm, to obtain the first blood pressure value of the to-be-measured user.

Further, the biological signal collection module 20 is specifically configured to determine whether the to-be-measured user is in a motionless state, and collect the first biological signal of the to-be-measured user in a preset collection period when the to-be-measured user is in a motionless state and wears a blood pressure monitoring device.

Optionally, the first biological signal, the second biological signal, and the third biological signals are all pulse wave signals of the to-be-measured user.

The blood pressure monitoring apparatus provided in this application can execute the foregoing method embodiment. Implementation principles and technical effects thereof are similar, and details are not described herein again.

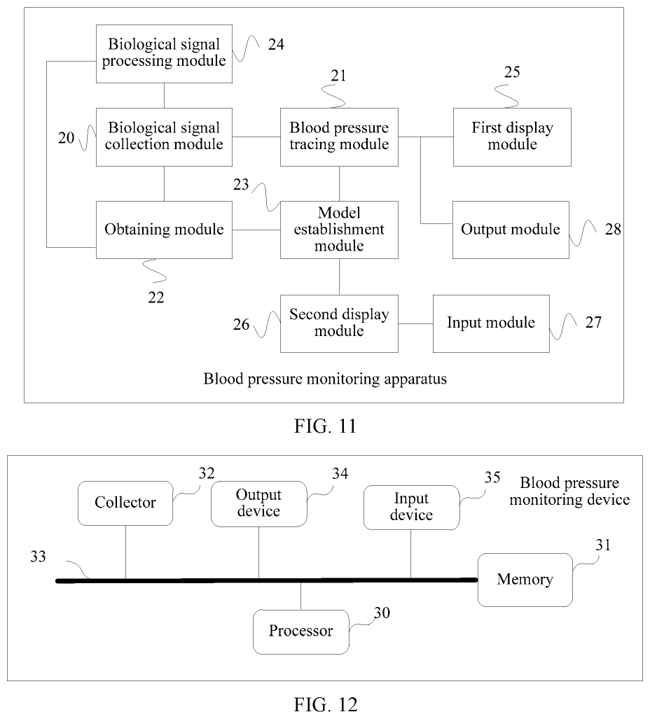

FIG. 11 is a schematic structural diagram of Embodiment 4 of a blood pressure monitoring apparatus according to this application. Based on the embodiment shown in FIG. 10, the apparatus further includes a first display module 25. Optionally, the apparatus may further include a second display module 26, an input module 27, and an output module 28.

The blood pressure tracing module 21 is configured to generate a blood pressure variation curve based on first blood pressure values predicted at different time points.

The first display module 25 is configured to display the blood pressure variation curve.

Optionally, the output module 28 is configured to output prompt information when the first blood pressure value of the to-be-measured user is greater than a preset threshold, where the prompt information is used to inform that blood pressure is abnormal.

Optionally, the input module 27 is configured to obtain a period setting operation entered by the to-be-measured user.

The second display module 26 is configured to display a period setting screen based on the period setting operation, where the period setting screen includes a plurality of model update periods.

The model establishment module 23 is configured to obtain the preset model update period based on a period selection operation of the to-be-measured user on the period setting screen.

The blood pressure monitoring apparatus provided in this application can execute the foregoing method embodiment. Implementation principles and technical effects thereof are similar, and details are not described herein again.

FIG. 12 is a schematic structural diagram of an embodiment of a blood pressure monitoring device according to the present invention. As shown in FIG. 12, the blood pressure monitoring device may include: a processor 30, for example, a CPU, a memory 31, a collector 32, and at least one communications bus 33. Optionally, the blood pressure monitoring device may further include an output device 34 and an input device 35. The communications bus 33 is configured to implement a communication connection between the components. The memory 31 may include a high-speed RAM memory, and may further include a non-volatile memory NVM, for example, at least one magnetic disk memory. The memory 31 may store various programs, to implement various processing functions and implement method steps of this embodiment. The collector 32 may be a device or an element having a biological signal collection function, or may be a device or an element having a biological signal collection function and a blood pressure collection function. For example, the collector may be a collection device for a pulse wave signal, or may be a device that can collect a pulse wave signal and that also includes elements such as a micropump, a pressure sensor, and an air pipe that are used for blood pressure collection. The output device 34 may be a voice output device, for example, a microphone or a speaker, or may be a display screen. The input device 35 is configured to provide an input interface for a user and receive an operation, an instruction, or the like that is entered by the user.

Specifically, in this embodiment, the collector 32 is configured to collect a first biological signal of a to-be-measured user.

The processor 30 is configured to predict a first blood pressure value of the to-be-measured user based on the first biological signal and a pre-established individual calibration model.

The individual calibration model is obtained based on calibration data of the to-be-measured user and preset model training data; the calibration data includes a second blood pressure value actually measured before the first biological signal of the to-be-measured user is collected and a second biological signal corresponding to the second blood pressure value; the model training data includes actually-measured third blood pressure values of training users and third biological signals corresponding to the third blood pressure values; and the first biological signal, the second biological signal, and the third biological signals are all physiological signals that can generate waveforms.

Further, the collector 32 is further configured to obtain at least one piece of calibration data of the to-be-measured user.

The processor 30 is further configured to establish, based on the at least one piece of calibration data and the model training data, the individual calibration model corresponding to the to-be-measured user.

Still further, the collector 32 is further configured to obtain at least one piece of new calibration data of the to-be-measured user when a preset model update period arrives.

The processor 30 is further configured to update the individual calibration model of the to-be-measured user based on the at least one piece of new calibration data, to obtain a new individual calibration model.

Optionally, the processor 30 is specifically configured to determine, in the model training data based on the at least one piece of calibration data, a training data set required by the to-be-measured user, and obtain, based on the training data set required by the to-be-measured user and a preset modeling algorithm, the individual calibration model corresponding to the to-be-measured user, where the individual calibration model is a parameter set including a plurality of model parameters.

Optionally, the processor 30 is specifically configured to perform a feature extraction operation on the first biological signal, to obtain a feature set that can represent the first biological signal, and calculate the feature values in the feature set and the model parameters in the parameter set based on a preset algorithm, to obtain the first blood pressure value of the to-be-measured user, where the feature set includes feature values arranged in a preset feature sequence, and feature values in different sequences represent different features of the first biological signal.

Optionally, the collector 32 is specifically configured to determine whether the to-be-measured user is in a motionless state, and collect the first biological signal of the to-be-measured user in a preset collection period when the to-be-measured user is in a motionless state and wears a blood pressure monitoring device.

Optionally, the first biological signal, the second biological signal, and the third biological signals are all pulse wave signals of the to-be-measured user.

Optionally, the processor 30 is further configured to generate a blood pressure variation curve based on first blood pressure values predicted at different time points. The output device 34 is configured to display the blood pressure variation curve.

Optionally, the output device 34 is further configured to output prompt information when the first blood pressure value of the to-be-measured user is greater than a preset threshold, where the prompt information is used to inform that blood pressure is abnormal.

Optionally, the input device 35 is configured to obtain a period setting operation entered by the to-be-measured user.

The output device 34 is configured to display a period setting screen based on the period setting operation, where the period setting screen includes a plurality of model update periods.

The processor 30 is further configured to obtain the preset model update period based on a period selection operation of the to-be-measured user on the period setting screen.

The blood pressure monitoring device provided in this application can execute the foregoing method embodiment. Implementation principles and technical effects thereof are similar, and details are not described herein again.

In addition, it should be noted that, correlated parts between the method embodiments of this application may be mutually referenced. The apparatus provided in each apparatus embodiment is configured to perform the method provided in the corresponding method embodiment. Therefore, for each apparatus embodiment, refer to the correlated part in the correlated method embodiment for understanding.

The name of the message/frame, module, or unit provided in the embodiments of this application is merely an example, and another name may be used, provided that a function of the message/frame, module, or unit is the same.

1. A blood pressure monitoring method, comprising:
obtaining, calibration data of the to-be-measured user, wherein the calibration data comprises a second blood pressure value and a second biological signal corresponding to the second blood pressure value;
establish, a pre-established individual calibration model based on the calibration data and preset model training data, wherein the preset model training data comprises a third blood pressure value of training users and a third biological signal corresponding to the third blood pressure value;
collecting a first biological signal of a to-be-measured user; and
predicting a first blood pressure value of the to-be-measured user based on the first biological signal and the pre-established individual calibration model, wherein the first biological signal, the second biological signal, and the third biological signals are physiological signals;
obtaining new calibration data of the to-be-measured user when a preset model update period arrives; and
establishing a new individual calibration model of the to-be-measured user based on the calibration data.

2. (canceled)

3. (canceled)

4. The method according to claim 1, wherein the establishing of a pre-established individual calibration model based on the calibration data and preset model training data comprises:
determining, a training data set required by the to-be-measured user in the model training data based on the calibration data; and
obtaining, based on the training data set required by the to-be-measured user and a preset modeling algorithm, the individual calibration model corresponding to the to-be-measured user, wherein the individual calibration model is a parameter set comprising a plurality of model parameters.

5. The method according to claim 4, wherein the predicting of a first blood pressure value of the to-be-measured user based on the first biological signal and a preset individual calibration model specifically comprises:
performing a feature extraction operation on the first biological signal, to obtain a feature set that can represent the first biological signal, wherein the feature set comprises feature values arranged in a preset feature sequence, and feature values in different sequences represent different features of the first biological signal; and
calculating the feature values in the feature set and the model parameters in the parameter set based on a preset algorithm, to obtain the first blood pressure value of the to-be-measured user.

6. The method according to claim 1 wherein the collecting of a first biological signal of a to-be-measured user comprises:
determining whether the to-be-measured user is in a motionless state; and
collecting the first biological signal of the to-be-measured user in a preset collection period when the to-be-measured user is in a motionless state and wears a blood pressure monitoring.

7. The method according to claim 1, wherein the first biological signal, the second biological signal, and the third biological signals are pulse wave signals.

8. The method according to claim 1, wherein the method further comprises:
generating a blood pressure variation curve based on first blood pressure values predicted at different time points; and
displaying the blood pressure variation curve.

9. The method according to claim 1, wherein the method further comprises:
outputting prompt information when the first blood pressure value of the to-be-measured user is greater than a preset threshold, wherein the prompt information is used to inform that blood pressure is abnormal.

10. The method according to claim 1, wherein the method further comprises:
obtaining a period setting operation entered by the to-be-measured user;
displaying a period setting screen based on the period setting operation, wherein the period setting screen comprises a plurality of model update periods; and
obtaining the preset model update period based on a period selection operation of the to-be-measured user on the period setting screen.

11-20. (canceled)

21. A blood pressure monitoring device, comprising a collector and a processor, wherein
the collector is configured to obtain calibration data of the to-be-measured user, wherein the calibration data comprises a second blood pressure value and a second biological signal corresponding to the second blood pressure value;
the processor is configured to establish a pre-established individual calibration model based on the calibration data and preset model training data, wherein the preset model training data comprises a third blood pressure value of training users and a third biological signal corresponding to the third blood pressure value;
the collector is configured to collect a first biological signal of a to-be-measured user; and
the processor is configured to predict a first blood pressure value of the to-be-measured user based on the first biological signal and a pre-established individual calibration model, wherein
the first biological signal, the second biological signal, and the third biological signals are all physiological signals.

22. (canceled)

23. (canceled)

24. The device according to claim 21, wherein the processor is configured to determine, in the model training data based on the calibration data, a training data set required by the to-be-measured user, and obtain, based on the training data set required by the to-be-measured user and a preset modeling algorithm, the individual calibration model corresponding to the to-be-measured user, wherein the individual calibration model is a parameter set comprising a plurality of model parameters.

25. The device according to claim 24, wherein the processor is configured to perform a feature extraction operation on the first biological signal, to obtain a feature set that can represent the first biological signal, and calculate the feature values in the feature set and the model parameters in the parameter set based on a preset algorithm, to obtain the first blood pressure value of the to-be-measured user, wherein the feature set comprises feature values arranged in a preset feature sequence, and feature values in different sequences represent different features of the first biological signal.

26. The device according to claim 21, wherein the collector is configured to determine whether the to-be-measured user is in a motionless state, and collect the first biological signal of the to-be-measured user in a preset collection period when the to-be-measured user is in a motionless state.

27. The device according to claim 21, wherein the first biological signal, the second biological signal, and the third biological signals are pulse wave signals.

28. The device according to claim 21, wherein the device further comprises an output device;
the processor is further configured to generate a blood pressure variation curve based on first blood pressure values predicted at different time points; and
the output device is configured to display the blood pressure variation curve.

29. The device according to claim 21, wherein the device further comprises an output device; and
the output device is configured to output prompt information when the first blood pressure value of the to-be-measured user is greater than a preset threshold, wherein the prompt information is used to inform that blood pressure is abnormal.

30. The device according to claim 21, wherein the device further comprises an input device and an output device;
the input device is configured to obtain a period setting operation entered by the to-be-measured user;
the output device is configured to display a period setting screen based on the period setting operation, wherein the period setting screen comprises a plurality of model update periods; and
the processor is further configured to obtain the preset model update period based on a period selection operation of the to-be-measured user on the period setting screen.
